(19) United States
(12) Patent Application Publication
(10) Pub. No.: US 2006/0215809 A1
(43) Pub. Date: Sep. 28, 2006

(54) System and method for motion and angulation profiles in tomosynthesis

(75) Inventors: Xianfeng Ni, Waukesha, WI (US); Baojun Li, Waukesha, WI (US); Xu Zhang, Brookfield, WI (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/088,019

(22) Filed: Mar. 22, 2005

(51) Int. Cl. H05G 1/60; A61B 6/00; G01N 23/00; G21K 1/12

(52) U.S. Cl. 378/21

(57) ABSTRACT

Certain embodiments of the present invention include methods and systems for improved motion and angulation profiles in tomosynthesis. A method includes designating a target with a first and second dimension. An x-ray beam is projected onto at least a portion of the target. The x-ray beam has an origin with a position along the first dimension. The x-ray beam also has a beam axis, a projection area, and an angle ∅ representative of an angular distance between the beam axis and the at least a portion of the target. The method further includes varying the angle ∅ based at least in part on the position of the origin along the first dimension. The angle ∅ is varied to substantially maintain the projection area.

### BACKGROUND OF THE INVENTION

The present invention relates generally to tomosynthesis. More specifically, the invention relates to angulation and motion profiles in a tomosynthesis system.

Tomography involves obtaining a two-dimensional image slice (or tomogram) from a three-dimensional volume. A variety of tomographic imaging techniques exist today, such as conventional linear tomography, computed axial tomography (CT), and positron emission tomography (PET).

A relatively new and promising tomographic imaging technique is tomosynthesis. Tomosynthesis allows retrospective reconstruction of an arbitrary number of tomographic planes of anatomies from a set of projection images acquired over a variety of angles. Compared to conventional linear tomography, tomosynthesis provides premium image quality and enhanced depth information at a lower x-ray dose. Image quality and depth information is, of course, important when diagnosing patients. Additionally, tomosynthesis is relatively fast and cost-effective.

FIG. 1 shows, generally, a tomosynthetic imaging system. In a tomosynthetic imaging system 10, a target 12 may be stationary while an x-ray source 14 moves along at least a first dimension 16. The first dimension 16 may be horizontal, vertical, or may be along any orientation useful for tomosynthetic imaging. In some configurations, the x-ray source 14 may move along two or more dimensions. For instance, the x-ray source 14 may move along an arc. As the x-ray source 14 moves, an x-ray beam 18 is projected towards the target 12 over a variety of angulations 20. The x-ray beam 18 originates, or has its origin at or near the x-ray source 14. The intersection of the x-ray beam 18 and a plane defined by the target 12 forms an x-ray projection area 28. The target 12 may be an x-ray detector. If an x-ray detector is used, the x-ray detector may be digital, in that the detector may generate digital images. Digital x-ray detectors have advantages over film-based detectors, but digital x-ray detectors may be relatively expensive.

Using the technique shown in FIG. 1, a series of x-ray projection images may be acquired over a variety of x-ray source angulations 20. The series of x-ray projection images may subsequently be processed with image processing techniques to reconstruct planar images. The resulting reconstructed planar images provide a high degree of clarity and structure resolution. The clarity and resolution may be attributed, in part, to information contained in the series of x-ray projection images acquired over a variety of angulations.

When performing x-ray imaging, it may be important to limit x-ray exposure. For instance, x-ray exposure on a person is presently regulated by the Food and Drug Administration as set forth in 21 C.F.R. 1020.30. It is therefore desirable to reduce excess x-rays in x-ray imaging systems.

For example, excess x-rays in tomosynthetic systems may result if part of the projection area 28 lands outside of the target 12. If the target is an x-ray detector, then x-rays that fall outside of the detector cannot be used for tomosynthetic imaging. Therefore, these x-rays may be considered unessential for tomosynthetic imaging.

The size of the x-ray beam 18 may be adjusted with a collimator. Certain collimators may be adjusted by electro-mechanical systems, for example. Some of these collimators have two or more moveable blades that adjust the x-ray beam size. One type of collimator has four blades. The blades may be moved to adjust a size of the x-ray beam 18. The adjusted x-ray beam cross-section may be a variety of rectangular shapes. Other shapes are possible as well. For instance, a four-blade collimator may form an x-ray beam cross-section into a polygon that has more than four sides, such as an octagon. The projection area 28 of an x-ray beam increases as the x-ray beam travels farther from an x-ray source. As a tomosynthesis system operates, a variety of projection area 28 sizes and shapes may be possible.

Because the size and shape of the x-ray beam projection area 28 varies during the operation of a tomosynthetic imaging system, it may be preferable to adjust the size of the x-ray beam 18 so that the projection area 28 falls substantially within a perimeter of the target 12. There may be at least two reasons for adjusting the x-ray beam in this manner. First, x-rays that do not fall on the target 12 may not be detected by the imaging system, and therefore may be unessential. Unessential x-rays increase the amount of x-ray dose received by an x-ray subject without increasing the performance of the system. Second, digital x-ray detectors may be relatively expensive. It may be preferable, therefore, to adjust the size of the x-ray beam 18 to efficiently use of the surface area of the target 12.

Thus, there is a need for a tomosynthetic imaging system that may adjust an x-ray beam according to movement of an x-ray source and angulations of an x-ray beam. Additionally, there is a need for a tomosynthetic imaging system that may adjust an x-ray beam such that the x-ray beam falls substantially within a target, such as a digital x-ray detector. Moreover, there is a need for a tomosynthetic imaging system that may adjust an x-ray beam to efficiently exploit a digital x-ray detector when used in combination with a tomosynthesis projection system.

### BRIEF SUMMARY OF THE INVENTION

Certain embodiments of the present invention provide a system and method for motion and angulation profiles in tomosynthesis. In an embodiment, a method of tomosynthesis includes designating a target. The target has a first and second dimensions. An x-ray beam is projected onto at least a portion of the target. The x-ray beam has an origin, and the origin has a position along the first dimension. The x-ray beam also has a beam axis, a projection area, and an angle ∅ that is representative of an angular distance between the target and the x-ray beam. The angle ∅ is varied based at least in part on the position of the origin along the first dimension. The angle ∅ is varied so that the x-ray beam projection area is substantially maintained.

In another embodiment, the method of tomosynthesis further includes varying angle ∅ based at least in part on a position of the target along the first dimension, a distance along a third dimension between the target and the origin, and a size of the target along a first dimension.

In another embodiment, the target of the method includes a digital x-ray detector. In yet another embodiment, the projection area remains substantially within the target. In yet another embodiment, the projection area substantially conforms to a size of the detector along the first dimension.

In an embodiment, a method of tomosynthesis includes identifying a target. The target has a first and second dimensions. An x-ray beam is projected onto at least a portion of the target. The x-ray beam has an source, and the source has a position along the first dimension. The x-ray beam also has a beam axis, a projection area, and an angle ∅ that is representative of an angular distance between the target and the x-ray beam. The x-ray beam also has an angle γ representative of the x-ray beam width along the first dimension. The x-ray source position is varied along the first dimension. The angle ∅ is varied based at least in part on the position of the source along the first dimension. The angle ∅ is varied so that the x-ray beam projection area is substantially maintained. The angle γ is varied based at least in part on the position of the source along the first dimension. The angle γ is varied so that the x-ray beam projection area is substantially maintained.

In another embodiment, the method of tomosynthesis further includes varying angle ∅ based at least in part on a position of the target along the first dimension, a distance along a third dimension between the target and the origin, and a size of the target along a first dimension.

In another embodiment, the method of tomosynthesis further includes varying angle γ based at least in part on a position of the target along the first dimension, a distance along a third dimension between the target and the origin, and a size of the target along a first dimension.

In another embodiment, the method may further include an x-ray beam with an angle α that represents the x-ray beam width along a second dimension. In yet another embodiment, the method may include the step of varying α based at least in part on angles ∅ and γ, so that the x-ray beam projection area is substantially maintained. In yet another embodiment the angle α is varied based at least in part on a size of the target along the second dimension, and a distance along a third dimension between the source and the target.

In another embodiment, the method may further include adjusting sizes l and w of the projection area which represent sizes along the first and second dimensions respectively. The size l may be adjusted with respect to angle γ and a source-to-image distance SID representing a distance between the source and the target. The size w may be adjusted with respect to angles ∅ and γ and size l. In yet another embodiment, the target includes a digital x-ray detector and the projection area remains substantially within the target.

In an embodiment, system for performing tomosynthesis is provided including an x-ray source capable of emitting an x-ray beam. The x-ray beam has a beam axis, a beam width, a projection area, and an angle γ representing the beam width along a first dimension. The system also has a target with a perimeter. Included is a first motion subsystem for moving the x-ray source along at least a portion of the first dimension to a first dimension position. Also, a second motion subsystem is provided for adjusting an angle ∅ representing an angular distance between the beam axis and the target. The system further includes at least one collimator capable of altering the angle γ based at least in part on the first dimension position of the x-ray source so that the projection area remains substantially within the perimeter of the target.

In another embodiment, the method of tomosynthesis further includes varying angle ∅ based at least in part on a position of the target along the first dimension, a distance along a third dimension between the target and the origin, and a size of the target along a first dimension.

In another embodiment, the method of tomosynthesis further includes varying angle γ based at least in part on a position of the target along the first dimension, a distance along a third dimension between the target and the origin, and a size of the target along a first dimension.

In another embodiment, the method may further include an x-ray beam with an angle α that represents the x-ray beam width along a second dimension. In yet another embodiment, the method may include the step of varying α based at least in part on angles ∅ and γ, so that the x-ray beam projection area is substantially maintained. In yet another embodiment the angle α is varied based at least in part on a size of the target along the second dimension, and a distance along a third dimension between the source and the target. In yet another embodiment the target includes a digital x-ray detector.

### BRIEF DESCRIPTION OF SEVERAL VIEWS OF THE DRAWINGS

FIG. 1 shows a tomosynthetic imaging system.

FIG. 2 shows x-ray beam geometry.

FIG. 3 shows x-ray beam geometry representative of a tomosynthetic imaging system used in accordance with an embodiment of the present invention.

FIG. 4 shows x-ray beam projection geometry representative of a tomosynthetic imaging system used in accordance with an embodiment of the present invention.

FIG. 5 shows a flow diagram for a method of tomosynthesis used in accordance with an embodiment of the present invention.

### DETAILED DESCRIPTION OF THE INVENTION

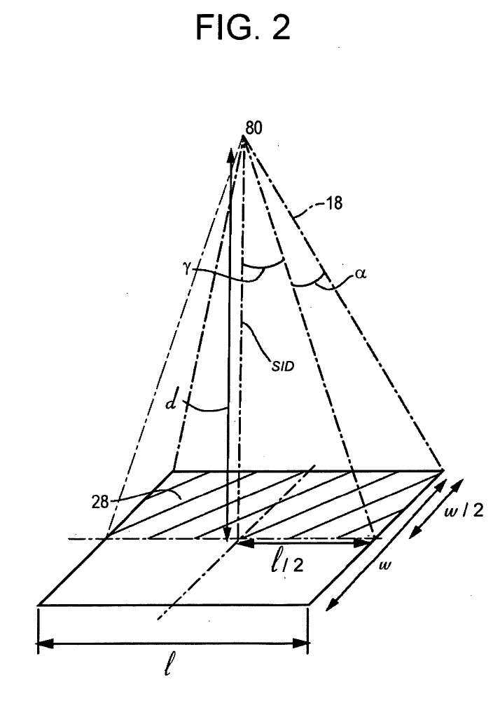

FIG. 2 shows a geometry representative of an x-ray beam 18. An x-ray beam 18 emanates from an origin 80. The x-ray beam 18 that has a projection area 28. FIG. 2 shows a rectangular projection area 28, but the x-ray beam projection area 28 may be in the shape of other geometric shapes, such as an octagon, a trapezoid, or a circle. The x-ray beam 18 also has a beam axis 34, and two beam width angles—α and γ. The projection area 28 has a width w and a length l. The beam width angles may be adjustable by a collimator, which is not shown in FIG. 2. As the x-ray beam 18 travels a distance Source-to-Image-Distance (SID) along a dimension d, the x-ray beam projection area 28 expands.

Equations (1) and (2) describe certain geometric relationships between α, γ, w, l, and SID:

$$\begin{matrix}tan ( γ ) = \frac{l}{2 \cdot SID} & ( 1 ) \\ tan ( α ) = \frac{w}{l} sin γ & ( 2 )\end{matrix}$$

Equations (1) and (2) may be solved for l and w respectively to arrive at equations (3) and (4):

$$\begin{matrix}l = 2 \times SID \times tan γ & ( 3 ) \\ w = \frac{l \times tan ( α )}{sin γ} & ( 4 )\end{matrix}$$

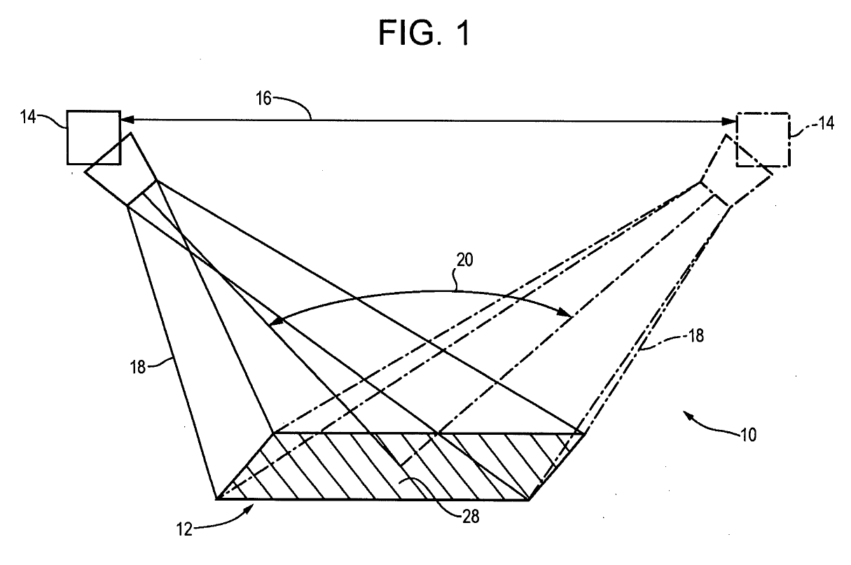

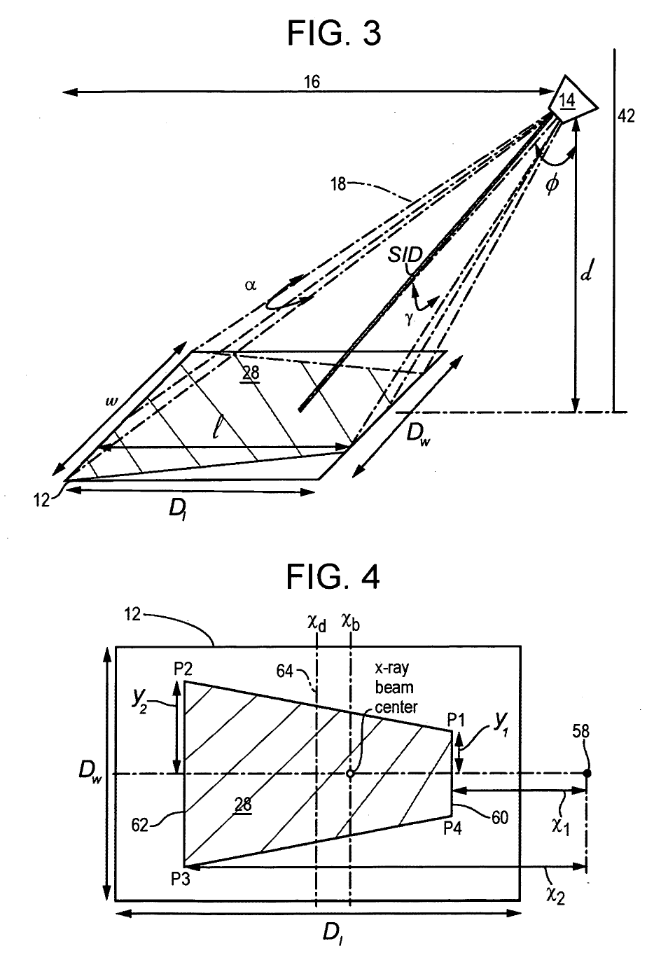

FIG. 3 shows a geometry representing tomosynthetic imaging systems. Referring for a moment to FIG. 1, the tomosynthetic imaging system 10 features an x-ray beam 18 that moves over a range of angulations 20 with reference to a stationary target 12. Turning back to FIG. 3, the angulation 20 may be represented by an angle ∅. Angle ∅ represents an angular distance between an x-ray beam axis 34 and a dimension 42 perpendicular to the x-ray detector, which may also be described as a third dimension.

When angle ∅ is not 0°, the x-ray beam 18 forms a projection area 28 that is trapezoidal. Moreover, as angle ∅ varies, and as the position of the x-ray source 14 varies along a first dimension 16, the projection area 28 will vary in size and shape.

Beam width angle γ represents an angular distance of the x-ray beam 18 along a first dimension. Beam width angle α represents an angular distance of the x-ray beam 18 along a second dimension. Distance d represents a distance between an x-ray source 14 and a position of the target 12 along a third dimension 42. A source-to-image distance SID represents a distance between the x-ray source and the target 12. The SID may be represented by the following geometric relationship:

$$SID = \frac{d}{cos ϕ} \qquad ( 5 )$$

FIG. 4 shows a geometry with respect to a first dimension and a second dimension used in accordance with an embodiment of the present invention. X-ray source position (x₂,yₛ) 58 is shown. The x-ray source position 58 also has a location along a third dimension 42 which is shown in FIG. 3, but not in FIG. 4. An x-ray beam projection area 28 is formed between the intersection of an x-ray beam 18 (shown in FIG. 3) and a plane defined by a target 12. The target 12 may be a digital x-ray detector, for example. When the x-ray beam 18 is rectangular, a trapezoidal projection area 28 is formed.

The projection area 28 has a first side 60 and a second side 62. As depicted in FIG. 3, the “fatter” side of the trapezoidal projection area 28 is on the second side 62. As angle ∅ varies from positive to negative (or vice versa), the trapezoidal projection area 28 changes shape as depicted in FIG. 3. A position x₁ represents a distance along a first dimension between the x-ray source position 58 and the first side 60 of the projection area 28. A position x₂ represents a distance along the first dimension between the x-ray source position 58 and the second side 62 of the projection area 28. A position X_{d} 64 represents a position along the first dimension of a center of the target 12. A size Dₗ represents a size of the target 12 along a first dimension, which may also be described as a length dimension. A size D_{w} represents a size of the target 12 along a second dimension, which may also be described as a width dimension. Looking at both FIGS. 3 and 4, the positions x₁ and X₂ may be represented by equation (6):

$$\{ \begin{matrix}{x}_{1} = d tan ( ϕ - γ ) \\ {x}_{2} = d tan ( ϕ + γ )\end{matrix} \qquad ( 6 )$$

A position y₁ represents a distance along the second dimension between the x-ray source position 58 and a corner of the first side 60 of the projection area 28. A position Y₂ represents a distance along the second dimension between the x-ray source position 58 and a corner of the second side 62 of the projection area 28. The positions y₁ and Y₂ may be described by equation (7):

$$\{ \begin{matrix}{y}_{1} = \frac{d}{cos ( ϕ - γ )} tan α \\ {y}_{2} = \frac{d}{cos ( ϕ + γ )} tan α\end{matrix} \qquad ( 7 )$$

In order for the x-ray beam projection area 28 to fall within the target 12 along a first dimension, the assumption represented by equation (8) may be used.

$$\{ \begin{matrix}{x}_{s} - {x}_{d} = {x}_{1} + \frac{{D}_{l}}{2} \\ {x}_{s} - {x}_{d} = {x}_{2} - \frac{{D}_{l}}{2}\end{matrix} \qquad ( 8 )$$

If the target 12 is, for instance, a digital x-ray detector, then the assumption represented by equation (8) has an additional advantage of efficiently utilizing the digital x-ray detector along a first dimension. In other words, the assumption in equation (8) represents an x-ray beam projection along a first dimension that substantially covers the digital x-ray detector along the first dimension.

Equation (9) may be obtained by solving equations (6) and (8) for angle ∅.

$$ϕ = \frac{1}{2} [ arctan \frac{( {x}_{s} - {x}_{d} + \frac{{D}_{l}}{2} )}{d} + arctan \frac{( {x}_{s} - {x}_{d} - \frac{{D}_{l}}{2} )}{d} ] \qquad ( 9 )$$

Equation (10) may be obtained by solving equations (6) and (8) for angle γ.

$$γ = \frac{1}{2} [ arctan \frac{( {x}_{s} - {x}_{d} + \frac{{D}_{l}}{2} )}{d} - arctan \frac{( {x}_{s} - {x}_{d} - \frac{{D}_{l}}{2} )}{d} ] \qquad ( 10 )$$

Equation (11) represents an angular velocity profile for angle ∅, and may be obtained by taking a derivative an angular position of angle ∅ as represented by equation (9).

$$\frac{ⅆ ϕ}{ⅆ t} = \frac{1}{2 d} \{ \begin{matrix}\frac{1}{1 + {( \frac{{x}_{s} - {x}_{d} + \frac{{D}_{l}}{2}}{d} )}^{2}} + \\ \frac{1}{1 + {( \frac{{x}_{s} - {x}_{d} - \frac{{D}_{l}}{2}}{d} )}^{2}}\end{matrix} \} \frac{ⅆ {x}_{s}}{ⅆ s} \qquad ( 11 )$$

Looking at FIGS. 3 and 4, notice that when angle ∅ is positive, y₁ is greater than Y2, and when when angle ∅ is negative, y₁ is less than Y_{2.} In other words, the “fatter” side of the trapezoid changes sides as angle ∅ goes from positive to negative, or from negative to positive. In order for the x-ray beam projection area 28 to fall within the target 12 along the second dimension, an assumption may be made: a maximum value of y₁ or Y₂ should not exceed half the size of the detector—or D_{w}/2. If the target 12 is, for instance, a digital x-ray detector, then it may be preferable to assume a maximum value of y₁ or y₂ equal to D_{w}/2. If a maximum value of y₁ or y₂ is substantially equal to D_{w}/2, a digital x-ray detector target may be efficiently utilized. Applying the above discussed assumptions to equation (7), the following equations (12), (13), and (14) may be obtained.

$$\begin{matrix}{D}_{w} / 2 = \frac{d}{cos ( \vert ϕ \vert - γ )} tan ( α ) & ( 12 ) \\ tan ( α ) = {D}_{w} \cdot cos ( \vert ϕ \vert - γ ) / ( 2 d ) & ( 13 ) \\ α = arctan ( {D}_{w} \cdot cos ( \vert ϕ \vert - γ ) / ( 2 d ) ) & ( 14 )\end{matrix}$$

The set of equations (9), (10), and (14) represent a set system angulations for ∅, α, and γ during tomosynthesis. By constraining a tomosynthesis system behavior to the assumptions represented by equations (8) and (12), the system angulations represented by equations (9), (10), and (14) may be derived.

A tomosynthesis system that adjusts system angulations for ∅, α, and γ based at least in part on equations (9), (10), and (14) may effectively project an x-ray beam projection area 28 on a target 12. Additionally, equations (9), (10), and (14) may assist a tomosynthesis system to project an x-ray beam projection area 28 such that the projection area 28 falls substantially within a target 12. If the target is, for instance, a digital x-ray detector, then equations (9), (10), and (14) may assist a tomosynthesis system to efficiently use the surface area of a digital x-ray detector.

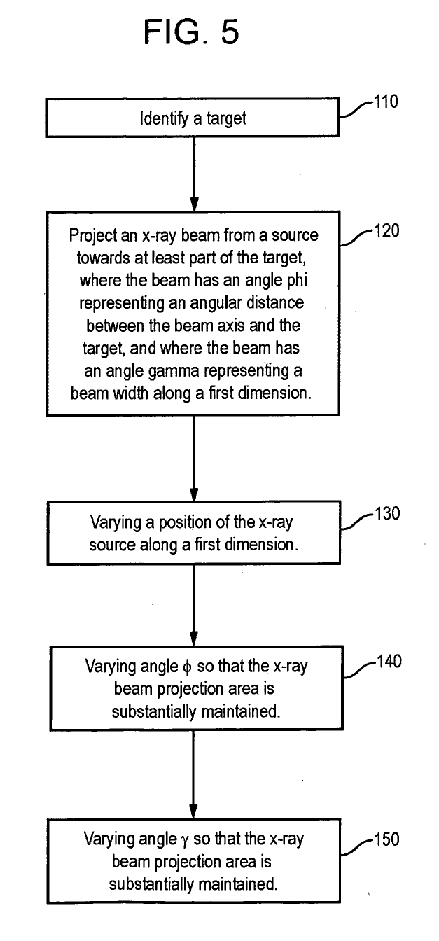

FIG. 5 shows a flow diagram for a method of tomosynthesis used in accordance with an embodiment of the present invention. At step 110, a target is identified. At step 120, an x-ray beam is projected from a source towards at least part of the target. The x-ray beam has an angle ∅ representing an angular distance between the x-ray beam axis and the target. The x-ray beam also has an angle γ representing a beam width angle along a first dimension. The x-ray beam may also have additional angles. At step 130, the x-ray source position is varied along the first dimension. At step 140, angle ∅ is varied so that the x-ray beam projection area is substantially maintained. At step 150, angle γ is varied so that the x-ray beam projection area is substantially maintained. Step 150 is optional.

The embodiments disclosed herein may also be useful to improve techniques for image pasting and auto-positioning. Image pasting is a technique for imaging an area larger than a detector size. In image pasting, a detector is movable. Different images are generated at various detector locations. The images are then pasted together. For example, using image pasting, it is possible to generate a single image of the whole spinal column using only a 41 cm square detector. As one with ordinary skill in the art would appreciate, the techniques disclosed herein may be useful for image pasting.

Auto-positioning is a technique that assists operators of x-ray imaging equipment to accurately position a tube source. Instead of manually positioning a tube source, auto-positioning may use motors or other automated motion subsystems to position the tube source in an appropriate location for imaging. As one with ordinary skill in the art would appreciate, the techniques disclosed herein may be useful for auto-positioning.

Thus certain embodiments provide a system and method for adjusting an x-ray beam according to movement of an x-ray source. Certain embodiments provide a system and method for adjusting an x-ray beam according to movement of an x-ray source and system angulations. Certain embodiments provide a system and method to adjust an x-ray beam to fall substantially within a target, such as a digital x-ray detector. Certain embodiments provide a system and method to adjust an x-ray beam to efficiently exploit the usable surface area of a target such as a digital x-ray detector. Certain embodiments may extend to other functions, such as image pasting, auto tracking, auto positioning, as well as field of view centering.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method of tomosynthesis comprising:
designating a target, said target comprising a first dimension and a second dimension;
projecting an x-ray beam onto at least a portion of said target, wherein said x-ray beam comprises an origin having a position along said first dimension, a beam axis, a projection area, and an angle ∅ representative of an angular distance between said beam axis and said first dimension of said at least a portion of said target; and
varying said angle ∅ based at least in part on said position along said first dimension of said origin to substantially maintain said projection area.

2. The method of claim 1 wherein
$$ϕ = \frac{1}{2} [ arctan \frac{( {x}_{s} - {x}_{d} + \frac{{D}_{l}}{2} )}{d} + arctan \frac{( {x}_{s} - {x}_{d} - \frac{{D}_{l}}{2} )}{d} ]$$
where xₛ represents said position of said origin along said first dimension, x_{d} represents a position of said target along said first dimension, d represents a distance along a third dimension between said origin and said target, and Dₗ represents a size along a said first dimension of said target.

3. The method of claim 1 wherein said target comprises a digital x-ray detector.

4. The method of claim 1 wherein said projection area remains substantially within said target.

5. The method of claim 1 wherein said projection area substantially conforms to Dₗ.

6. A method of tomosynthesis comprising:
identifying a target, said target comprising a first dimension and a second dimension;
projecting an x-ray beam onto at least a portion of said target, said x-ray beam having a source, a beam axis, a projection area, an angle ∅ representing an angular distance between said beam axis and said first dimension of said at least a portion of said target, and an angle γ representing an x-ray beam width along said first dimension;
varying a position of said x-ray source along at least said first dimension; and
varying said angle γ and said angle ∅ based at least in part on said position along said first dimension of said x-ray source to substantially maintain said projection area.

7. The method of claim 6 wherein
$$ϕ = \frac{1}{2} [ arctan \frac{( {x}_{s} - {x}_{d} + \frac{{D}_{l}}{2} )}{d} + arctan \frac{( {x}_{s} - {x}_{d} - \frac{{D}_{l}}{2} )}{d} ] .$$
where xₛ represents said position of said along said first dimension, X_{d} represents a position of said target along said first dimension, d represents a distance along a third dimension between said origin and said target, and Dₗ represents a size along said first dimension of said target.

8. The method of claim 6 wherein
$$γ = \frac{1}{2} [ arctan \frac{( {x}_{s} - {x}_{d} + \frac{{D}_{l}}{2} )}{d} - arctan \frac{( {x}_{s} - {x}_{d} - \frac{{D}_{l}}{2} )}{d} ] .$$
where x₂ represents said position of said along said first dimension, X_{d} represents a position of said target along said first dimension, d represents a distance along a third dimension between said origin and said target, and Dₗ represents a size along a said first dimension of said target.

9. The method of claim 6 wherein said x-ray beam further comprises an angle α representing an x-ray beam width along said second dimension.

10. The method of claim 9 further comprising the step of varying said angle α based at least in part on said angle ∅ and said angle γ to substantially maintain said projection area.

11. The method of claim 10 wherein
α=arctan (D_{w}.cos(|∅|−γ)/(2d))
where D_{w} represents a size along a said second dimension of said target, and d represents a distance along a third dimension between said source and said target.

12. The method of claim 6 wherein
l=2×SID×tan γ
where SID represents a distance between said x-ray source and said target, and l represents a size of said projection area along said first dimension.

13. The method of claim 6 wherein
$$w = \frac{l \times tan ( α )}{sin γ}$$
where l represents a size of said projection area along said first dimension, and w represents a size of said projection area along said second dimension.

14. The method of claim 6 wherein said target comprises a digital x-ray detector, and said projection area remains substantially within said target.

15. A system for performing tomosynthesis comprising:
an x-ray source capable of emitting an x-ray beam, said x-ray beam having a beam axis, a beam width, a projection area, and an angle γ representing said beam width along a first dimension;
a target with a perimeter;
a first motion subsystem for moving said x-ray source along at least a portion of said first dimension to a first dimension position;
a second motion subsystem for altering an angle of said tube source thereby adjusting an angle ∅ representing an angular distance between said beam axis and said first dimension; and
at least one collimator capable of altering said angle γ, wherein said altering is based at least in part on said first dimension position of said x-ray source so that said projection area remains substantially within said perimeter of said target.

16. The system of claim 15 wherein
$$ϕ = \frac{1}{2} [ arctan \frac{( {x}_{s} - {x}_{d} + \frac{{D}_{l}}{2} )}{d} + arctan \frac{( {x}_{s} - {x}_{d} - \frac{{D}_{l}}{2} )}{d} ]$$
where xₛ represents said position of said x-ray source along said first dimension, X_{d} represents a position of said target along said first dimension, d represents a distance along a third dimension between said x-ray source and said target, and Dₗ represents a size along said first dimension of said target.

17. The system of claim 15 wherein
$$γ = \frac{1}{2} [ arctan \frac{( {x}_{s} - {x}_{d} + \frac{{D}_{l}}{2} )}{d} - arctan \frac{( {x}_{s} - {x}_{d} - \frac{{D}_{l}}{2} )}{d} ] .$$
where xₛ represents said position of said x-ray source along said first dimension, X_{d} represents a position of said target along said first dimension, d represents a distance along a third dimension between said x-ray source and said target, and Dₗ represents a size along a said of said target.

18. The system of claim 15 wherein said x-ray beam further comprises angle α representing said beam width along said second dimension;
and wherein in said at least one collimator is further capable of altering an angle α based at least in part on said angle ∅ and said angle γ.

19. The system of claim 15 wherein
α=arctan(D_{w}.cos(|∅|−γ)/(2d))
where D_{w} represents a size along a said second dimension of said target, and d represents a distance along a third dimension between said origin and said target.

20. The system of claim 15 wherein said target comprises a digital x-ray detector.
